(19) United States
(12) Patent Application Publication
(10) Pub. No.: US 2022/0176093 A1
(43) Pub. Date: Jun. 9, 2022

(54) SYSTEM FOR THE PERCUTANEOUS APPLICATION OF A FLUID TO SKIN AREAS

(71) Applicants: Yara Reichert, Buch am Erlbach (DE); Esther Probst, Hohenschaeftlarn (DE)

(72) Inventors: Yara Reichert, Buch am Erlbach (DE); Esther Probst, Hohenschaeftlarn (DE)

(21) Appl. No.: 17/437,032

(22) PCT Filed: Mar. 5, 2020
(86) PCT No.: PCT/DE2020/100149

§ 371 Date: Sep. 7, 2021

(30) Foreign Application Priority Data
Mar. 8, 2019 (DE) 20 2019 101 350.8
May 13, 2019 (DE) 20 2019 102 669.3

(51) Int. Cl. A61M 35/00; A61M 37/00

(52) U.S. Cl. CPC A61M 35/10; A61M 35/30; A61M 2210/0606; A61M 2037/0007; A61M 37/00

(57) ABSTRACT

A system for the percutaneous application of a fluid to one or more surface areas of a skin. The system may include at least one of at least one compressor and at least one fan for providing compressed air. The system may also include an applicator fluidically connectable to the at least one of the at least one compressor and the at least one fan. The applicator may have at least one outlet opening through which a fluidic jet is flowable and directable onto the skin. The at least one of the at least one compressor and the at least one fan is attached to the applicator.

### CROSS-REFERENCE TO RELATED APPLICATIONS

This application claims priority to International Patent Application No. PCT/DE2020/100149, filed on Mar. 5, 2020, German Patent Application No. 202019101350.8, filed on Mar. 8, 2019, and German Patent Application No. 202019102669.3, filed on May 13, 2019, the contents of all of which are hereby incorporated by reference in their entirety.

### TECHNICAL FIELD

The present invention relates to a system for the percutaneous application of a fluid to skin areas. The fluid can be gaseous, for example, air, a gas mixture or pure oxygen. However, the fluid can also be a sprayable composition with gaseous, fluid and viscous components, a cream, a gel or an aerosol, and contain a pharmaceutical or cosmetic active substance.

### BACKGROUND

For numerous medical and also cosmetic applications, it is desirable to apply an active substance via the skin until it penetrates deeply into tissue.

The skin consists of a plurality of layers; it is delimited to the outside by the epidermis, a keratinizing, multi-layered squamous epithelium which is free from blood vessels and has an outer horny layer. The dermis, which has a blood supply, is located under the squamous epithelium. The subcutis, the subcutaneous fatty tissue, is located under the skin. The barrier function of the skin is brought about above all by the epidermis. The barrier function of the epidermis proves to be an obstacle in respect of the transdermal transport of active substances, whether pharmaceutical active substances or cosmetic active substances. Conventional cosmetic and pharmaceutical preparations are scarcely capable of overcoming the epidermis barrier. The active substance virtually does not penetrate deeper skin layers. In order to penetrate deeper skin layers, a liquid active substance can be injected with an injection needle into the skin. It is also possible to use what are referred to as jet injections without an injection needle to inject a liquid active substance under high pressure through the skin into the body with the aid of a strongly focused jet.

These methods are not suitable for extensive use on surface areas of the skin and also not for the improved supply of gaseous oxygen to deeper skin layers.

In principle, most skin regions of the human body can be treated by the application of a fluid. For the treatment of the facial skin, special masks are known which, on the side facing the face, have a multiplicity of openings through which a fluid can be directed under pressure onto areas of the facial skin.

One such mask for treating the facial skin is known, for example, from Swiss Patent CH 168278. The face mask described there for therapeutic purposes has two mask parts which lie one inside the other and are tightly interconnected and of which the outer is closed and the inner is perforated, and means for conducting a massage medium through the mask in such a manner that the skin is massaged by a multiplicity of inwardly directed jets. The therapy here consists of a massage. A percutaneous application of an active substance into deeper skin layers is clearly not conceived of here.

US patent U.S. Pat. No. 3,088,459 discloses a therapeutic cosmetic mask with which a fluid, generally water, can be sprayed onto the facial skin via a plurality of nozzles, the fluid being able to be either heated or cooled in order to open skin pores to facilitate the penetration of an active substance, or to close the pores by means of cooled water and to wash off the residue of the active substance from the skin.

Furthermore, a massage mask operating with rotating nozzles has been disclosed in DE 103 48 614 B3. The mask serves exclusively for massaging the facial skin, penetration of active substances into the skin is not conceived of.

For the treatment of the skin in other body regions, handheld devices are known from the prior art which can conduct at least one jet of pressurized fluid onto the skin. Such devices serve, for example, for treating cellulitis on the thigh skin by introducing an active substance into deeper layers of the skin. Such an application system is known, for example, from DE 10 2010 044 674 B4.

### SUMMARY

The present invention is based on the object of presenting a system for the percutaneous application of a fluid, said system avoiding the above-described disadvantages of the prior art, and of presenting an application system for the treatment of skin areas, the application system being easy to handle and, within a short period, permitting a pharmaceutical or cosmetic treatment of skin areas, with which active substances can be effectively introduced into deeper regions of the skin.

This object is achieved with a system for the percutaneous application of a fluid to skin areas, the system having the features of the independent claim(s).

Dependent claims are directed toward preferred embodiments of the present invention.

The application system according to the invention furthermore has an applicator which is fluidically connectable at least to the compressor or the fan and has at least one outlet opening through which a fluid jet can emerge which can be directed onto the skin.

The applicator can also be adapted to the shape of specific locations of the body, for example in the form of a face mask which is fastenable to the head of the user.

According to one embodiment of the invention, the compressor or the fan is attached directly to the applicator. This simplifies the handling of the system which can thereby be used independently of location.

In contrast to stationary systems, for example in salons or practices, in which percutaneous applicators are connected to stationary pressure systems which are arranged next to a treatment chair, the system according to the invention can be used everywhere and can furthermore be carried in luggage because of its compactness.

The applicator can be a rigid face mask for attaching in front of the face, the face mask having a multiplicity of outlet openings which are distributed over the inner side facing the face and are fluidically connected at least to the compressor or the fan. The mask can also be configured to be flexible in order to be better adapted to the shape of the face.

A pressure regulating chamber can be connected downstream of the compressor or fan, and a multiplicity of outlet nozzles for the fluid that are directed onto the skin to be treated are fluidically connected to the pressure regulating chamber.

Electrically switchable valves which are controlled by a valve controller, or else manually actuable valves can be arranged in the fluid connections between the pressure regulating chamber and the outlet nozzles. It is thus possible to reduce the number of outlet nozzles which are charged with the pressure medium and/or the fluid containing an active substance in order to avoid an excessive drop of pressure at the output of the outlet nozzles.

The face mask can be formed with a single wall, with channels having outlet openings or nozzles directed at the skin being arranged on its side facing the face.

The face mask can be designed with double walls, with the pressure regulating chamber being able to be arranged, for example, between the inner side facing the face and an outer side facing away from the face.

The positive pressure generated by the compressor or fan at the outlet nozzles or outlet openings can be between 1 and 8 bar, preferably between 2 and 5 bar.

The face mask can have an opening at the lower nose region for breathing purposes and, in the eye region, can provide protection of the eyes, for example in the form of ribs or beads of a soft, skin-friendly material which protrude toward the face and prevent the pressure medium and/or the fluid from entering the eyes. In the eye region, the mask can contain openings, and it can be provided to arrange the outlet nozzles not too close to the eye region such that the system can remain open.

The outlet nozzles can be arranged obliquely in relation to the inner wall of the mask and can be mounted rotatably, and also can have a turbine wheel such that they execute a rotational movement because of the pressurization, and therefore the treatment region of each nozzle on the facial skin is enlarged. The circulating movement of the nozzles also results in a certain massage effect.

In a further embodiment of the present invention, it is provided to apply the cosmetic or pharmaceutical active substance in the form of a cream, a gel or the like to the locations of the skin to be treated, and then subsequently to drive the active substance into deeper layers of the skin with the applicator in the form of a mask or in the form of a handheld device.

A plurality of classes of active substances can be used. These include purely cosmetic products, pharmaceutical products and products which are used in medical therapy, such as, for example, cancer therapy.

All of the active substances listed in tables 1 to 3 on pages 5 to 23 of EP 1 819 317 B1 are suitable as the active substances which can be applied with the system according to the invention for the percutaneous application of a fluid to surface areas of the skin. The disclosure of this European patent is expressly incorporated here; in particular, active substances which contain vitamin C or edelweiss extract are particularly suitable.

The applicator, whether the face mask or the handheld device, in addition to the outlet nozzles of the pressure medium and/or the fluid, can have LEDs for simultaneous action upon the skin to be treated with light of certain selected frequencies. The compressor or the fan can be fastened to the face mask in the upper region, for example can be fastened to the mask via the forehead of a user. Further preferred attachment locations are located directly below the nose region or in the chin region. Additional compressors or fans can in each case be attached laterally.

In addition, the compressor or the plurality of compressors or the fan or the plurality of fans can be connected to the face mask or the handheld device via a short hose such that the user can sit, for example, at a table and can place the compressor or the fan, or a plurality thereof, in front of themself on the table, and therefore the facemask or the handheld device are lighter, which is of benefit to the comfort during a treatment.

According to the invention, that part of the face mask which faces the face can be adapted individually to the shape of the face of a user. For this purpose, according to the invention, the shape of the face of the user is detected in a 3D scanner and the data thus obtained, for example Sketchfab® data of the user, are supplied to a 3D printer with which an individually adapted inner side of the mask can be manufactured.

The controllable valves preferably allow the pressure medium and/or fluid to flow out continuously during a treatment period. However, the controllable valves can also allow the pressure medium and/or fluid to emerge in a pulsed manner.

With the valve controller, it is possible to activate valves of certain groups of outlet nozzles in order to treat certain face regions separately from one another. In this case, the face regions to be treated can be sequentially selected automatically by the valve controller. It is also possible for all face regions to be treated simultaneously as long as the pressure of the pressure medium is of a sufficient magnitude.

According to a further embodiment of the present invention, the face regions to be treated can be selected manually via a keypad on the outer side of the mask.

The face mask preferably has flexible beads in the chin region and along the lower jaw and either in the region of the root of the nose or on or over the forehead in order to keep the face mask at a distance from the facial skin. The face mask is fastened by a headband at its upper edge to the head, and the compressor or the fan are fastened together with the pressure regulating chamber to the upper side of the face mask.

According to a preferred embodiment of the invention, the compressor or the fan together with the pressure regulating chamber is arranged in the lower region of the mask below the nose. The compressor or the fan can also be fastened together with the pressure regulating chamber to the mask below the chin region of the latter.

Furthermore, additional compressors or fans can be arranged with the pressure regulating chamber on the side edges of the mask and/or at other unoccupied points of the mask.

According to a preferred embodiment of the invention, the face mask has interchangeable attachments having in each case a different arrangement and/or configuration of outlet openings in order to be able to treat individual face regions individually. Valves and/or openings in partial regions of the mask and/or of the interchangeable attachments separate from one another can be controlled individually here.

The handheld device can also be fitted with different interchangeable attachments which have different outlet openings or valves.

The pressure at the output of the compressor or of the fan can advantageously be adjustable in at least three stages—small, medium, large—via a switch.

The compressed air and/or the fluid can be guided through a filter, wherein said filter advantageously contains activated carbon.

The compressed air emerging from the compressor or fan can be heated or cooled by a heating/cooling system.

Furthermore, the applicator head and/or an interchangeable attachment can be set to vibrate by a vibrator for a massage function of the handheld device.

### BRIEF DESCRIPTION OF THE DRAWINGS

The present invention will be explained in more detail below using the description of exemplary embodiments with reference to the drawing, in which:

FIG. 1 shows a cross section through human skin;

FIG. 2 shows an exemplary embodiment of a facemask according to the invention with a holding strap and compressor or fan;

FIG. 3 shows an exploded view of an application system according to the invention with a face mask and compressor or fan;

FIG. 4 shows a further embodiment of the application system with a mask and compressor or fan;

FIG. 5 shows a variant of the application system shown in FIG. 4; and

FIG. 6 shows a handheld application system according to the present invention.

### DETAILED DESCRIPTION

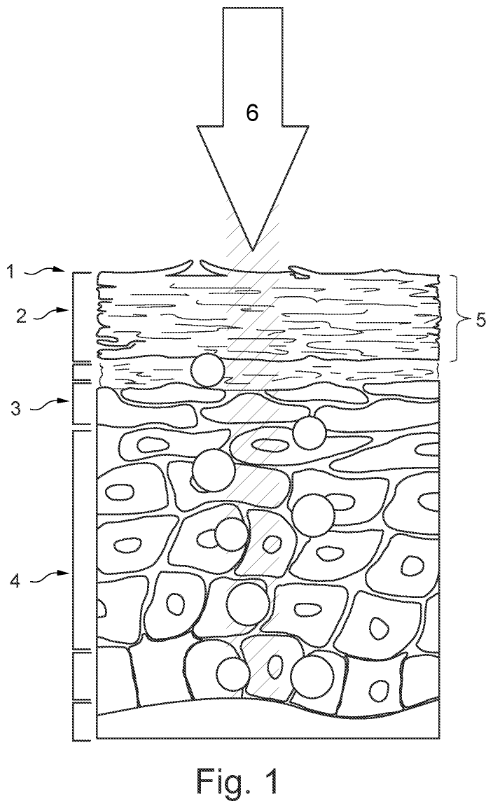

As FIG. 1 shows, the human skin is outwardly sealed by an outer horny layer, followed by a keratinizing, multi-layered squamous epithelium layer free from blood vessels. The dermis having a blood supply is located underneath. The subcutaneous fatty tissue is located under the skin. The horny layer and the squamous epithelium form the epidermis. By means of a jet of a pressurized fluid which, in addition to the compressed air, can also contain added active substances, the pressure medium, which can be, for example, compressed air or oxygen-enriched compressed air, pure oxygen or another gaseous substances, is injected, optionally together with particles of a liquid or viscous active substance, into the skin, and this can take place as far as the subcutis, depending on the pressurization. Thus, for example, the skin can be enriched with oxygen as far as deep layers, and active substances which can be added to the propellant, such as, for example, vitamin C and/or edelweiss extract, can also be applied as far as deep layers of the skin in order to deploy their positive effect there for the complexion.

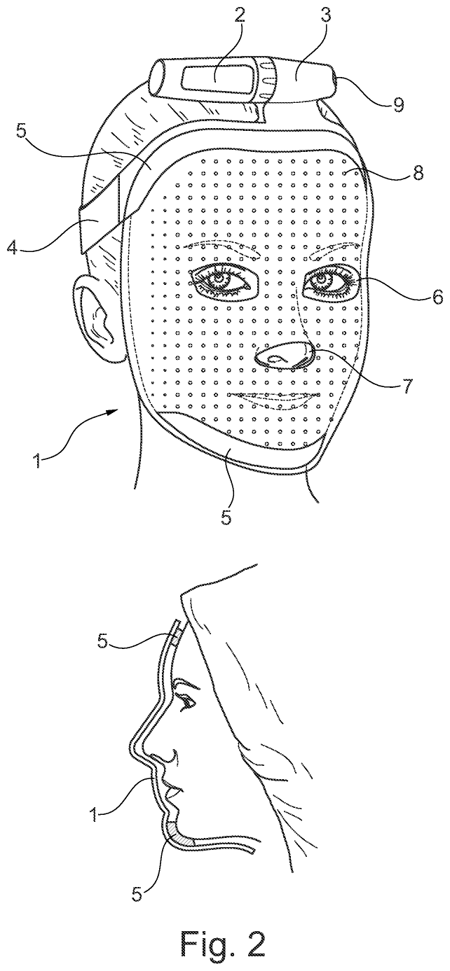

FIG. 2 shows a first embodiment of a system according to the invention for the percutaneous application of a fluid with a facemask 1 and a compressor or a fan 2, which is arranged on the upper edge of the mask 1 and is fluidically connected to the face mask via a pressure regulator 3. The mask 1 is fastened to the head with an elastic headband 4.

The mask has skin contact bands 5 in the chin region and in the upper forehead region, via the thickness of which the distance of the inner side of the mask from the facial skin is determined. Said distance can expediently be in the range between 5 and 15 mm. The face mask shown in FIG. 2 has an outer shell which is airtight and an inner shell which is perforated with a predetermined pattern of openings through which the pressure medium and/or the fluid emerges from the mask and strikes against the facial skin. Openings are provided in the region of the eyes and the nose, the inner walls of which openings reach from the outer shell as the far as the inner shell in order to avoid pressure medium escaping from the intermediate spaces between the two mask shells.

In certain regions, the inner shell can have removable interchangeable attachments which are interchangeable and which have a different positioning of outlet openings or outlet nozzles depending on the desired application. Such regions can be, for example, the temple region, the cheeks or the forehead region.

If it is desired to provide, for example, a more intensive treatment of the temple regions, an interchangeable attachment can be exchanged in the temple region and replaced by an interchangeable attachment with a higher opening density.

According to one variant embodiment of the present invention, the user of the application system can first of all apply a desired active substance in the form of a cream, a gel, a liquid, an aerosol or the like to the locations of the skin to be treated in the face, and then to attach the face mask and to activate the compressor or the fan 2 together with the pressure regulator 3 by actuation of a switch 9 via which an outlet valve of the compressor or fan 2 is opened. The outlet valve can be an electrically controlled valve which is connected to a control logic which is activated by the switch 9. The control logic can open the compressor valve or fan valve in a continuous or pulsating manner.

The pressure regulator 3 can furthermore be adjusted via a manual switch at least in the stages small, medium and large.

The pressure regulator 3 has a pressure regulating chamber in which the pressure medium emerging from the compressor valve or fan valve is reduced to a suitable pressure with which the pressure medium enters the intermediate space between the mask shells. The pressure medium can be compressed in the compressor 2 to a pressure of the order of magnitude of 10 bar, and said pressure is reduced via the pressure regulator 3 until the pressure of the pressure medium/fluid emerging from the openings 8 is reduced from 1 to 5 bar.

In addition, to the compressor or fan, the applicator can also have a cartridge from which gases or aerosols under high pressure can be added. For example, compressed mountain air can be involved.

In one specific embodiment, the compressor 2 is operated by battery or storage battery. One such compressor is sold, for example, by TOPTREK®. This compressor is substantially cylindrical, with a length of approx. 15 cm and a diameter of 2.5 cm. It supplies a maximum pressure of 6-7 bar and one storage battery charge is sufficient for a treatment duration of approx. 10 minutes. Without the storage battery, the compressor has a weight of approximately 80 grams.

In order to be able to increase the operating duration of a treatment, instead of a storage battery, the compressor or the fan can be fed with mains power via a power cable.

A fan which is suitable for the use descried here is sold by Guédon. It is likewise substantially cylindrical and weighs approx. 70 g. With this fan, a pressure of 3 bar can be generated on the output side. This fan is also fed by a storage battery with a nominal voltage of 12 V.

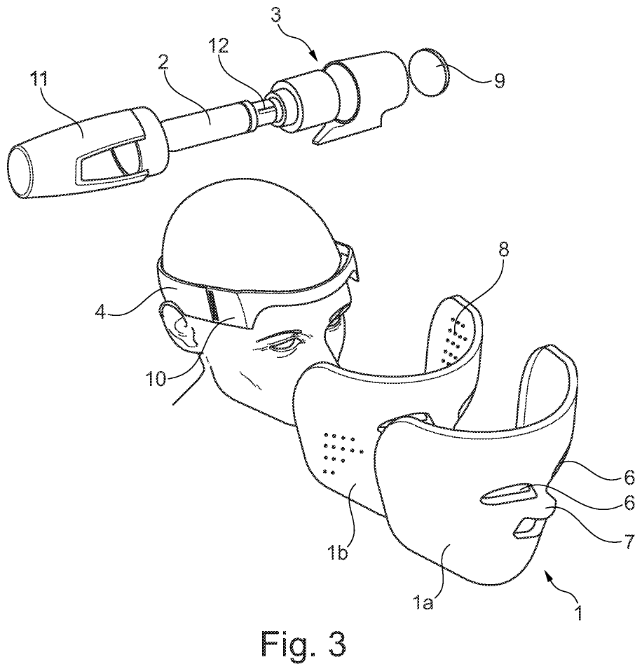

FIG. 3 shows an exploded view of the components of the application system shown in FIG. 2. The elastic headband 4 has touch and close strips 10 in each case in the temple region, via which the mask 1 can be fastened on the head.

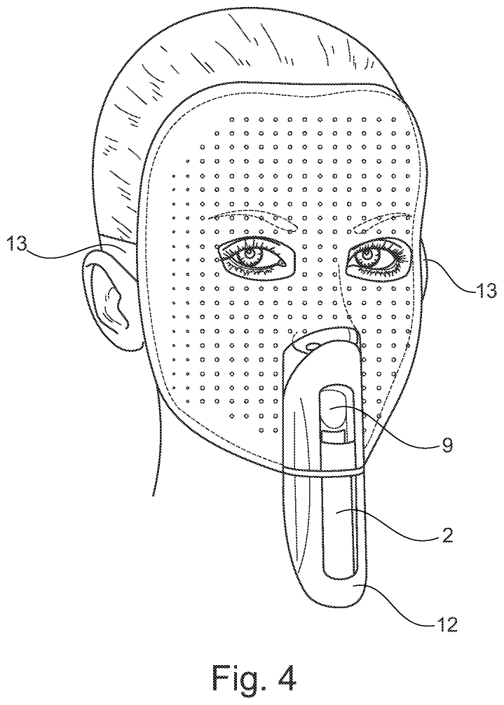

FIG. 4 shows a variant of the application system schematically, wherein some of the features shown in FIGS. 2 and 3 have been omitted. In this variant, the compressor or the fan and the pressure regulator 3 are arranged in the region below the nose of the mask, which simplifies the handling of the application system. By gripping the compressor housing or fan housing 12, the position of the mask during the use thereof on the face of the user can be additionally stabilized.

FIG. 4 furthermore shows a variant of the fastening of the mask on the head, which, as shown here, takes place via ear hooks 13. The actuating button for activating the compressor/fan 2/pressure regulator 3 is located at a location of the pressure regulator 3 that can easily be reached by the index finger of the gripping hand.

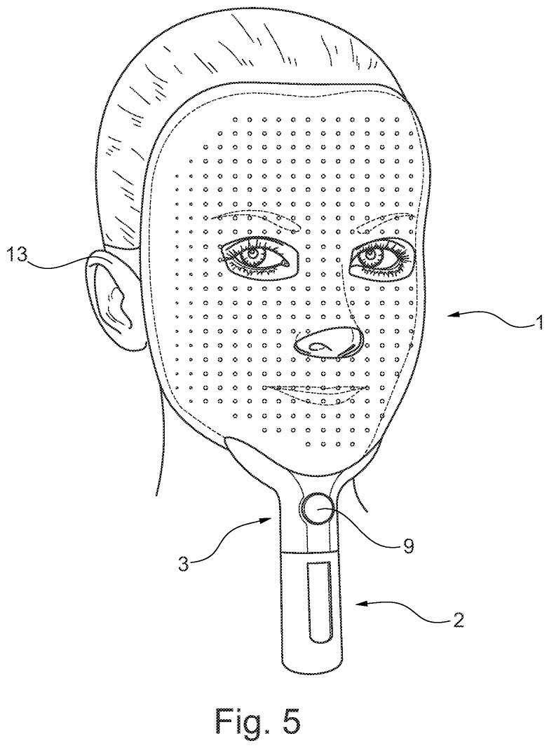

Finally, FIG. 5 shows a further variant of the application system according to the invention in which the compressor or the fan 2 and the pressure regulator 3 are arranged on the lower side of the mask 1 in the chin region. As a result, the center of gravity of the application system can be lowered even further and can be displaced in the direction of the user's neck, which further assists the stable seating of the mask 1 on the user's head.

At the lower end of the compressor or fan 2, a power cable with anti-kink protection can be led out of the housing if the compressor or the fan 2 is fed with mains power.

In an alternative embodiment, not shown, the compressor or the fan 2 can also be designed as a separate part which is connected to the pressure regulator 3 via a hose and can thus be set down, for example, on a table at which the user of the system sits, as a result of which the mask becomes overall lighter, which is beneficial for the comfort of wearing same.

The pressure regulator 3 together with the compressor or fan can also form a unit which is separate from the face mask, and the connecting hose then connects the output of the pressure regulator to an inlet nozzle on the face mask.

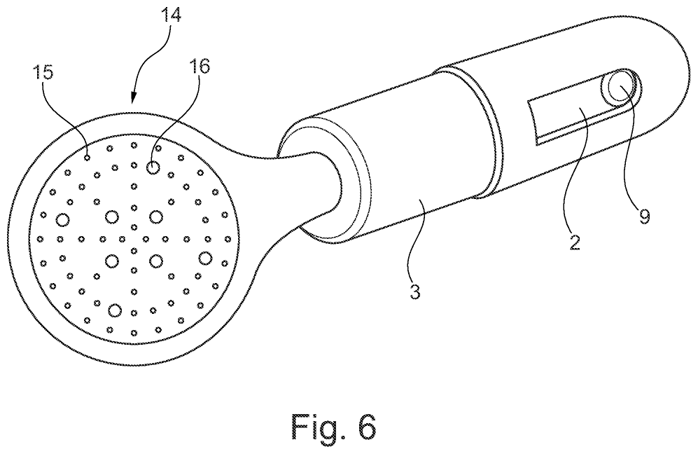

FIG. 6 shows an embodiment of an application system according to the invention in the form of a handheld device. The compressor or the fan 2 and a pressure regulating chamber 3 are installed there in the handle of the handheld device. The pressure regulating chamber is adjoined by the treatment head which substantially has the form of a showerhead. That is to say, on one side of the treatment head 14 openings or nozzles are provided out of which the pressure medium and/or the fluid emerges and can be directed onto the skin of a location of the body. In the embodiment shown in FIG. 6, LEDs are furthermore attached between the nozzles or openings for simultaneously illuminating the skin with light of a certain wavelength. The percutaneous application of a fluid can thus be combined simultaneously with light therapy.

Also in the case of the handheld device, the compressor or the fan 2 can be designed separately and connected to the applicator head via a hose. Similarly, in this case, the pressure regulator can be fastened either to the applicator head or to the compressor or fan 2.

Furthermore, a vibrator can be arranged in the handheld device at a suitable location, for example at the applicator head or at an interchangeable attachment, in order to provide the handheld device with an additional massage function.

1. A system for the percutaneous application of a fluid to one or more surface areas of a skin, the system comprising:
at least one of at least one compressor and at least one fan for providing compressed air;
an applicator fluidically connectable to the at least one of the at least one compressor and the at least one fan, the applicator having at least one outlet opening through which a fluidic jet is flowable and directable onto the skin; and
wherein the at least one of the at least one compressor and the at least one fan is attached to the applicator.

2. The system as claimed in claim 1, wherein:
the applicator is structured as a face mask for attaching in front of a face;
the face mask includes at least one of a plurality of outlet openings and a plurality of outlet nozzles distributed over an inner side of the face mask configured to face the face, the plurality of outlet openings including the at least one outlet opening; and
the at least one of the plurality of outlet openings and the plurality of outlet nozzles are fluidically connected at least to the at least one of the at least one compressor and the at least one fan.

3. The system as claimed in claim 1, wherein the applicator together with the at least one of the at least one compressor and the at least one fan are structured as a handheld device, which is guidable with one hand to a plurality of locations to be treated on the skin.

4. The system as claimed in claim 2, wherein a positive pressure provided by the at least one of the at least one compressor and the at least one fan at the at least one of the plurality of outlet nozzles and the plurality of outlet openings is from 1 to 8 bar.

5. The system as claimed in claim 2, wherein the face mask has a lower nose region, an opening disposed at the lower nose region for breathing purposes, and an eye region that is kept open.

6. The system as claimed in claim 2, wherein:
the face mask includes the plurality of outlet nozzles;
the plurality of outlet nozzles each have an outlet end of a plurality of outlet ends; and
the plurality of outlet nozzles are arranged obliquely in relation to an inner wall of the face mask, are mounted rotatably, and are driven to perform a rotational movement by a pressure medium emerging therefrom via a turbine wheel such that the plurality of outlet ends each execute a circular movement.

7. The system as claimed in claim 6, wherein a radius of the circular movement is dimensioned such that an impact surface of an outlet cone of the pressure medium on the skin defines a closed full-surface circle.

8. The system as claimed in claim 1, wherein the at least one of the at least one compressor and the at least one fan has an electric motor and is fed by at least one of a storage battery, a battery, and a mains power via a cable.

9. The system as claimed in claim 2, further comprising an active substance to be applied percutaneously as at least one of a liquid, an aerosol, a cream, a paste, and a gel to an area of the skin to be treated, and wherein the active substance is drivable into a plurality of intracellular intermediate spaces of an epidermis of the skin via a pressure of a pressure medium.

10. The system as claimed in claim 2, wherein the face mask includes a plurality of interchangeable attachments each having a different configuration of the at least one of the plurality of outlet openings and the plurality of outlet nozzles such that a plurality of individual face regions of the face are individually treatable.

11. The system as claimed in claim 10, wherein the face mask further includes a plurality of valves that are individually controllable such that the at least one of the plurality of outlet nozzles and the plurality of outlet openings in a plurality of partial regions of at least one of the face mask and the plurality of interchangeable attachments separate from one another are individually controllable.

12. The system as claimed in claim 3, wherein:
the at least one outlet opening includes a plurality of outlet openings;
the handheld device is fittable with a plurality of different interchangeable attachments each having a different configuration of the plurality of outlet openings; and
a pressure at an output of the at least one of the at least one compressor and the at least one fan is adjustable via a switch to at least 3 stages including a small stage, a medium stage, and a large stage.

13. The system as claimed in claim 1, further comprising a heating/cooling system configured to at least one of heat and cool the compressed air provided by the at least one of the at least one compressor and the at least one fan.

14. The system as claimed in claim 1, further comprising a filter through which the compressed air is guidable.

15. The system as claimed in claim 12, further comprising a vibrator, wherein at least one of an applicator head of the applicator and the plurality of interchangeable attachments are vibratable via the vibrator to provide a massage function.

16. The system as claimed in claim 1, further comprising a hose connecting the applicator and the at least one of the at least one compressor and the at least one fan.

17. The system as claimed in claim 2, wherein the face mask has an eye region and a plurality of ribs disposed in the eye region, the plurality of ribs protruding towards the face and configured to shield a user's eyes from a treatment region of the face mask.

18. The system as claimed in claim 2, further comprising:
an elastic headband;
a plurality of contact bands configured to rest on the face and set a distance between the face and the face mask;
wherein a first contact band of the plurality of contact bands is arranged in an upper forehead region of the face mask; and
wherein a second contact band of the plurality of contact bands is arranged in a chin region of the face mask.

19. The system as claimed in claim 4, wherein the positive pressure is from 2 to 5 bar.

20. The system as claimed in claim 10, wherein:
the face mask includes an outer shell and an inner shell;
the inner shell includes the plurality of interchangeable attachments; and
the plurality of interchangeable attachments are arranged in a temple region, a cheek region, and a forehead region of the face mask.
